(19) United States
(12) Patent Application Publication
(10) Pub. No.: US 2019/0314640 A1
(43) Pub. Date: Oct. 17, 2019

(54) WOUND TREATMENT

(71) Applicant: Steve Mencanin, Lake Forest, CA (US)

(72) Inventor: Steve Mencanin, Lake Forest, CA (US)

(21) Appl. No.: 16/356,354

(22) Filed: Mar. 18, 2019

Related U.S. Application Data
(60) Provisional application No. 62/644,404, filed on Mar. 17, 2018.

(51) Int. Cl. A61N 5/06; A61F 13/00

(52) U.S. Cl. CPC A61N 5/0616; A61F 13/00051; A61N 2005/0659; A61N 2005/0651; A61N 2005/0645

(57) ABSTRACT

The present invention relates to methods and use of electromagnetic radiation for the treatment of wounds, especially superficial and deep wounds and ulcerations.

### CROSS REFERENCE TO RELATED APPLICATION

This application claims the benefit of U.S. Provisional Application Ser. No. 62/644,404, filed Mar. 17, 2018, the entire disclosure of which is incorporated herein by reference.

### FIELD OF THE INVENTION

The present invention relates to the use of electromagnetic radiation for the treatment of wounds, especially superficial and deep wounds and ulcerations.

### BACKGROUND

Chronic wounds represent a significant burden to patients, health care professionals, and the health care systems.

Various non-surgical approaches have been developed and numerous drugs have been introduced to aid the management of wounds. For example, compression bandages, medicated bandages, intermittent pneumatic compression and vacuum assisted closure can be used, usually in combination with medicaments. As regards therapeutics it is known to use agents that improve perfusion of peripheral blood vessels, prostacyclin analogues, antimicrobials, nitric acid donors, calcium agonists, steroids, antioxidants and agents that increase angiogenesis, collagen synthesis and epithelialisation.

Other non-surgical approaches have been advocated in the treatment of chronic wounds based on methods of improving circulation and thus oxygen and nutrient delivery to a wound site to improve healing times. This may be achieved mechanically by, for example, radiant heat dressing, ultrasound therapy, laser treatment, hydrotherapy, oxygen therapy, electrotherapy, electromagnetic therapy, and PUVA therapy (psoralen plus ultraviolet A irradiation). The prevailing wisdom is that thermal action on a wound site promotes the healing process by increasing localised blood flow.

Despite great strides in technological innovations and the emergence of a wide range of treatments for wounds, non-healing wounds continue to perplex and challenge doctors.

There is a need for alternative, effective and safe methods of wound healing.

There is a need for a simple self-administered wound healing treatment.

### SUMMARY OF THE DISCLOSURE

According to a first aspect of the invention there is provided use of electromagnetic radiation centred around 1072 nm for the treatment of wound healing.

Preferably, the electromagnetic radiation is divergent light between 10° to 50°. By divergent it is meant that the electromagnetic radiation emitted from the electromagnetic source has a divergent half angle of at least 5°. Preferably divergence of the electromagnetic radiation is in the range 15° to 25° half angled divergent.

Thus it will be appreciated that the method of the present invention does not include the use of lasers as the source of electromagnetic radiation.

It has now been surprisingly established that low intensity electromagnetic radiation of small bandwidth (preferably around 10 nm to 120 nm and more preferably 50 nm) is effective in treating wounds. It is postulated that the way in which the electromagnetic radiation effects its action is by way of energy transmission through cellular components/organelles, enzymes such as but not limited to inducible nitric oxide synthase (iNOS). A water molecule that has a range of electromagnetic radiation wavelengths passed through it will produce several transmission peaks. These transmission peaks are associated with the preferred therapeutic electromagnetic radiation wavelength range of the invention and thus implies a role for the water molecule in the general mechanism of action.

Our studies have shown that wavelengths centred around those wavelengths specified above and especially around single restricted bandwidth light centred at 1072 nm or 1267 nm are particularly effective at reducing wrinkle length and area. It is of note that these two wavelengths correspond to the peak emission wavelengths of a water molecule light transmission profile and thus we believe that the mechanism of action is related to water and possibly cell membranes.

Furthermore, our studies have also shown that 1072 nm and 880 nm elicit opposing effects upon lymphocyte viability ex vivo, the former being protective and the latter wavelength cytotoxic. Furthermore, electromagnetic radiation centred around 1072 nm protects against UV-mediated lymphotoxicity.

Preferably, the electromagnetic radiation is continuous or pulsed.

Preferably, when the electromagnetic radiation is continuous the intensity is at least 500 μWatts/cm² and up to 500 mWatts/cm². Preferably when the electromagnetic radiation is pulsed the intensity is at least 500 μWatts/cm² peak power and the average power is up to 500 mWatts/cm². The average power is the peak power multiplied by the proportion of the total time that the radiation is applied. For instance, if the peak power is 500 μWatts/cm² and is pulsed for 10 μseconds at a frequency of 600 Hz then the average power is 30 μWatts/cm².

Prior art methods which rely on thermal warming specify a lower limit of 0.5 Watts/cm² the present invention which seeks to avoid any thermal effects operates below this level.

Preferably when the electromagnetic radiation is pulsed the average power of the intensity is in the region of 50-100 p Watts/cm².

We have found that the power may suitably range from 500 μWatts/cm² peak to 500 mWatts/cm² continuous or peak power when applied to the skin. Typically, 20 mWatts/cm² are used on skin but this value is dependent on how fat or muscular the subject is and thus how deep the wound lies.

Preferably when the electromagnetic radiation is pulsed it is applied for periods of at least 10-15 μseconds and more preferably is applied at a frequency/repetition rate in the range 300-900 Hz more preferably still the frequency/repetition rate is at, or about, 600 Hz.

Our studies have shown that the electromagnetic radiation can be either coherent or non-coherent the clinical outcomes are not affected by this parameter.

Preferably the electromagnetic radiation is applied to the affected area for at least 3 to 20 minutes. A typical exposure time is in the region of 5 to 10 minutes, however for deeper wounds this time is increased according to the individual's fat layer depth and exposure could be up to 15 minutes.

Preferably, the electromagnetic radiation is applied to the wound site at least one a day over a period of days, weeks or months depending on the severity of the wound site. It is expected that a treatment protocol would include a treatment several times per day such as 2, 3 4, 5, 6 times per day.

It should be appreciated that the power source emitting the electromagnetic radiation will have to produce more than the required intensity for the clinical effect since we have shown that approximately 99% of the applied therapeutic amount of light is lost across the skin surface during treatment. Thus the intensity of applied radiation will have to be corrected for when carrying out a treatment.

From the foregoing it is understood that the electromagnetic radiation may be directed to the wound site either continuously or in a switched (pulsed) manner. The main benefit of switching enables power conservation and facilities much higher peak power output, thereby improving wound healing response.

Preferably, the electromagnetic radiation therapy source includes means for reducing the amount of ambient radiation, which impinges on the treatment site.

Preferably, the electromagnetic radiation source is a light emitting diode. The radiation from such devices can be electrically operated or the radiation can be delivered to an applicator via a fibre-optic delivery system.

Preferably, the radiation source emitter includes a PN junction arranged to emit radiation with a wavelength centring at or about the previously mentioned specified wavelengths. A single light diode assembly may include a plurality of orientated junctions. Infrared emitting diodes may be arranged not only to emit radiation at a specific frequency but also to emit a high intensity divergent beam. The divergent light may also be derived from light emitting polymers.

The present invention is concerned with a method of managing wound treatment with divergent electromagnetic radiation having a wavelength centred around 1072 nm and optionally also 1267 nm. The electromagnetic radiation is applied at a low intensity such that no thermal damage or heating is caused to the skin or any other tissue or organ around the treatment area. In this way, the method of the present invention differs from the prior art as the effects are non-thermal and avoid thermolysis.

According to a second aspect of the invention there is provided a method wound treatment, the method comprising exposing a wound site to electromagnetic radiation centred around 1072 nm.

According to a third aspect of the invention there is provided a method of reducing time taken for a wound to heal, the method comprising exposing a wound site to electromagnetic radiation centred around 1072 nm.

According to a fourth aspect of the invention there is provided a method of reducing scarring of a wound, the method comprising exposing a wound site to electromagnetic radiation centred around 1072 nm.

We have found using the method of the present invention that elastin fibres become less fragmented and more uniform hence improve the elastic characteristics of the wound site and hence reduce scarring.

According to a fifth aspect of the invention there is provided a method of reducing pain associated with a wound site, the method comprising exposing a wound site to electromagnetic radiation centred around 1072 nm.

According to a sixth of the invention there is provided a method of treating diabetic ulcers, the method comprising exposing an ulceration site to electromagnetic radiation centred around 1072 nm.

Preferably, the second, third, fourth, fifth and sixth aspects of the invention further include any one or more of the features hereinbefore described of the first aspect of the invention. Light therapy, both laser and LED, have been shown to provide clinical benefit in many therapeutic arenas. The effects of light centred around wavelengths of 1072 nm and 880 nm, using a range of single and multiple irradiation protocols, have been assessed for their in vitro effect on freshly prepared human lymphocytes stimulated with phytohemagglutinin (PHA). Viable cell numbers remained significantly higher after irradiation with 1072 nm and were significantly lower after 880 nm irradiation compared to untreated controls, following a daily single irradiation over a five-day period. In addition, cell numbers were significantly higher after pre-treatment with 1072 nm and exposure to UVA, compared to cells treated with UVA only. Cells irradiated twice on Day 3 post-harvest with various wavebands confirm on Day 5, an increase in % cell viability after exposure to 1072 nm, and 1072 nm alternating with 1268 nm irradiation, and a decrease in % cell viability after 880 nm irradiation alone.

According to a further aspect of the invention there is provided device comprising a flexible substrate comprising a number of light emitting means that produce divergent electromagnetic radiation centred around 1072 nm, the substrate is provided with a number of apertures through which the light passes.

Preferably, the substrate may be contoured around a part of the body requiring treatment.

Preferably, the substrate is woven or non-woven. In one embodiment it is a bandage in another embodiment it may be a padded plastic panel.

Preferably, the device includes a power means which may be battery or mains electricity. Preferably, the light emitting device is a LED or more preferably a plurality of LEDs. It will be appreciated that the device of the present invention is not a laser device.

### BRIEF DESCRIPTION OF THE DRAWINGS

Embodiments of the invention are further described hereinafter with reference to the accompanying drawings, in which:

FIG. 1 shows a chronic leg wound of a patient having had leg surgery for squamous cell carcinoma removal.

FIG. 2 shows the same patient following treatment with electromagnetic radiation centred around 1072 nm.

### DETAILED DESCRIPTION

“Wound” as used throughout the specification and claims includes superficial wounds, deep wounds, ulceration and burns and means damage to one or more tissues, including open wounds such as cuts, scrapes, surgical incisions and the like, both acute and chronic, as well as internal wounds, for example, bruises, haematomas, fascia or soft tissue damage, hernia, abdominal wall damage, and the like as well as burns. “Wounds” may refer to tissue damage that occurs by trauma, or damage to tissue resulting from pathological sequelae, for example ischemia, that is exhibited due to overlying disease. The present invention is particularly useful in the healing of chronic open wounds such as ulcers, and diabetic ulcers in particular.

Reference hereinto “improving wound healing” is intended to include improving the time taken for a wound to heal, reducing amount of scarring of the wound, improving tissue tone and elasticity of the tissue and skin at the wound site, reducing reliance on therapeutic agents and reducing pain at the wound site.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### EXAMPLE 1

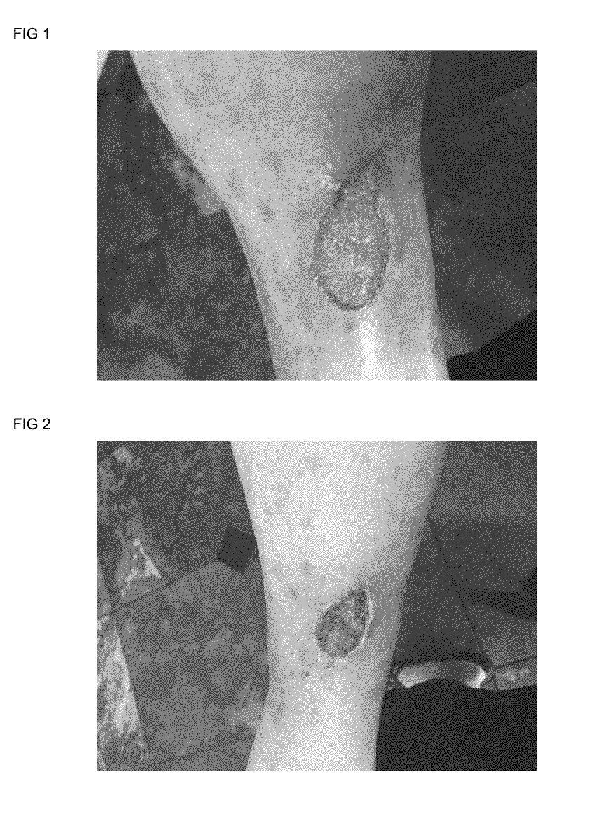

The patient had been on oral and topical antibiotics as well as platelet rich plasma [PRP] injections for almost 5 months after a large squamous cell carcinoma was removed from an area that had previous surgeries. The patient was having a difficult, slow healing process (FIG. 1) and refused a graft. The patient continued with both topical antibiotics, using them at the same time as the electromagnetic light treatment. The patient was instructed to use the electromagnetic light treatment over the entire wound for about 9 min 2-3 times a day. Positive changes were seen with the addition of the electromagnetic light treatment over 5 days (FIG. 2) to the extent of scabbing, reduced wound exudate and scab shrinkage. Without wishing to be bound by theory, it is postulated that increased blood flow in the affected area along with increased immune responses caused by the electromagnetic light treatment assisted in the wound healing process.

1-14. (canceled)

15. A method of wound treatment, the method comprising exposing a wound site to electromagnetic radiation centred around 1072 nm.

16-20. (canceled)

21. A device comprising a flexible substrate comprising a number of light emitting means that produce divergent electromagnetic radiation centred around 1072 nm, the substrate being provided with a number of apertures through which the light passes.

22. A device according to claim 21 wherein the substrate is capable of being contoured around a part of the body requiring treatment.

23. A device according to claim 21 wherein the substrate is woven or non-woven.

24. A device according to claim wherein the substrate is a bandage.

25. A device according to claim 21 wherein the substrate is a padded plastic panel.

26-27. (canceled)

28. A method according to claim 15 wherein the wound is a superficial or deep wound or ulceration.

29. A method according to claim 15 wherein the wound treatment is selected from the group consisting of reducing time taken for the wound to heal, reducing scarring of the wound, reducing pain associated with the wound.

30. A method according to claim 15 wherein the electromagnetic radiation is divergent light between 10° to 50°.

31. A method according to claim 1 wherein the electromagnetic radiation comprises a bandwidth of between 10 nm to 120 nm.

32. A method according claim 1 wherein the electromagnetic radiation is continuous or pulsed.

33. A method according to claim 1 wherein when the electromagnetic radiation is continuous the intensity is at least 500 μWatts/cm² and up to 500 mWatts/cm².

34. A method according to claim 1 wherein when the electromagnetic radiation is pulsed the intensity is at least 500 μWatts/cm² peak power and the average power is up to 500 mWatts/cm².

35. A method according to claim 34 wherein when the electromagnetic radiation average power of intensity is in the region of 50-100 mWatts/cm².

36. A method according to claim 34 wherein the pulsed electromagnetic radiation it is applied for periods of at least 10-15 μseconds.

37. A method according to claim 36 wherein frequency/repetition rate is in the range 300-900 Hz.

38. A method according to claim 15 wherein the electromagnetic radiation is applied to an affected area for at least 3 to 20 minutes.

39. A method according to claim 15 wherein the electromagnetic radiation is applied to the wound site at least one a day over a period of days, weeks or months until the wound has healed.

40. A method according to claim 15 wherein the electromagnetic radiation source is a light emitting diode.

41. A method according to claim 40 wherein the radiation source emitter includes a PN junction arranged to emit radiation with a wavelength centring at or about 1072 nm.
